(19) United States
(12) Patent Application Publication
(10) Pub. No.: US 2012/0134941 A1
(43) Pub. Date: May 31, 2012

(54) METHOD FOR SMOOTHING OUT THE SKIN BY FILLING IN RECESSED PORTIONS

(75) Inventors: Guillaume Cassin, Villebon Sur Yvette (FR); Henri Samain, Bievres (FR); Jean-Thierry Simonnet, Cachan (FR)

(73) Assignee: L'Oreal, Paris (FR)

(21) Appl. No.: 13/382,438

(22) PCT Filed: Jun. 29, 2010
(86) PCT No.: PCT/EP2010/059173

§ 371 Date: Feb. 3, 2012

Related U.S. Application Data
(60) Provisional application No. 61/242,016, filed on Sep. 14, 2009.

(30) Foreign Application Priority Data
Jul. 7, 2009 (FR) 0954673

(51) Int. Cl. A61K 8/81; A61Q 19/08; A61Q 17/04; A61Q 19/00; A61K 8/88; A61K 8/73

(52) U.S. Cl. 424/59; 424/78.03; 514/57

(57) ABSTRACT

The invention relates to a method for smoothing out the skin by filling in recessed portions of the skin, said method comprising filling the recessed portions by means of a filling-in product which has an elastic modulus of greater than 20 000 Pascals at 25° C. for a stress frequency of 1 Hertz, a flow point of greater than 500 Pascals, and, for applied stresses of less than the yield stress, a viscosity of greater than 100000 Pa.s, the product being such that, once applied in said recessed portions of the skin, its mechanical properties change subsequent to a reaction other than an evaporation reaction or a laser polymerization re action.

The present invention relates to masking wrinkles, and more generally to smoothing the surface of the skin by filling in the recessed regions such as wrinkles, pores or folds.

The presence of wrinkles on the face often poses aesthetic problems which, depending on the individual, can be difficult to live with.

The main component of the problem lies in the visibility of these wrinkles. Several cases are distinguished:

Wrinkles that will be termed “marked”: they are very visible and can show a depth of 300 microns or more.

Wrinkles that will be termed “medium”: they are visible and their depth ranges between 150 microns and 300 microns.

Wrinkles that will be termed “slight”: they are less visible than the previous wrinkles and their depth is less than 150 microns.

Wrinkles are also distinguished according to their location. Some wrinkles are located in very visible areas, for instance the area around the eyes. Others are located in less visible areas, for instance on the top of the forehead, which can generally be masked by the hair.

Wrinkles which come from skin ageing are distinguished from those which are not the result of ageing. The first are not present on young faces and are therefore recognized as signs of ageing. They are the wrinkles which appear around the eyes, between the eyebrows, on the forehead and on the cheeks. The second are present on young faces and are therefore tacitly recognized as natural. They are the folds which form a border with the cheeks. It will be noted that these folds often become more marked during ageing, and it is advantageous to take them into consideration.

There are mainly four types of solutions that exist at the current time.

The first comprises covering the wrinkled area with a fluid that will be able to mask the wrinkles. Several approaches have been developed for giving the area a hazy appearance and, with the optical contrast being decreased, reducing the visibility of the wrinkles. This solution has several limits: it is effective only on slight wrinkles. The effect is not long-lasting. In general, it is necessary to recommence the treatment within the next few hours.

This approach has been worked on by decreasing the thickness of the covering layer. However, as visualized on Scheme 3, a good result is not obtained on the wrinkles.

Admittedly, the skin keeps a natural appearance and the demarcations are not very visible, but the wrinkles are just covered and not filled in.

The second solution comprises treating the skin of the wrinkles. Several active agents are known for their activity on skin ageing. Mention may be made of: retinol and derivatives thereof, such as retinyl palmitate; ascorbic acid and derivatives thereof, such as magnesium ascorbyl phosphate and ascorbyl glucoside; adenosine and derivatives thereof, in particular non-phosphated derivatives; tocopherol and derivatives thereof, such as tocopheryl acetate; nicotinic acid and precursors thereof, such as nicotinamide; ubiquinone; glutathione and precursors thereof, such as L-2-oxothiazolidine-4-carboxylic acid; C-glycoside compounds and derivatives thereof, in particular those described in application WO 02/051828; plant extracts, and in particular extracts of sea fennel and of olive leaf, and also plant proteins and hydrolysates thereof, such as rice or soya protein hydrolysates; algal extracts and in particular of laminaria; bacterial extracts; sapogenins, such as diosgenin and extracts of Dioscorea plants, in particular of wild yam, containing them; α-hydroxy acids; β-hydroxy acids, such as salicylic acid and 5-n-octanoylsalicylic acid, oligopeptides and pseudodipeptides and acyl derivatives thereof, in particular {2-[acetyl(3-trifluoromethylphenyl)amino]-3-methylbutyrylamino}acetic acid and the lipopeptides sold by the company Sederma under the trade names Matrixyl 500 and Matrixyl 3000; lycopene; manganese salts and magnesium salts, in particular gluconates; and mixtures thereof. Mention may also be made of vitamins, such as, for example, vitamins B3 or PP, B5, E and K1.

These active agents make it possible to reduce the depth of the wrinkles, in general by causing preferential desquamation. However, as in the previous case, it is effective only on slight wrinkles. Admittedly, more powerful treatments are possible, but, given their effects on the skin, it is difficult to use them regularly or to make them available to everyone. In addition, problems of discomfort may appear.

The third solution comprises covering the area of the wrinkles with a fluid composition containing a “tensioning” material. Said material has the property of retracting during drying, and thus causing a tensile force at the limits of the area covered. The tensile force can then pull the wrinkled skin and reduce the visibility of the wrinkles.

This approach has several limits: it is effective only on slight wrinkles. In order to be really effective, it is important to use thick, highly tensioning materials. Under these conditions, the treatment can bring about quite a considerable reduction in the wrinkles, but it causes discomfort which limits its use. The effect is not long-lasting. It can also make it possible to reduce the visibility of medium wrinkles, but in this case, it is far from being completely effective.

The fourth solution comprises treating the subcutaneous muscles, called skin muscles.

According to a first approach, the skin muscles are treated by paralysing them. Thus, it is possible to relax the wrinkles and to make them disappear, at least in part. It is effective for wrinkles formed around the eyes, the forehead and the mouth.

Even though it has the advantage of lasting, this approach has several drawbacks which greatly reduce its development: it is painful since, in order to reach the skin muscles, it is necessary to inject the paralysing product. Thus, it is suitable only for highly motivated individuals. It is not therefore suitable for individuals who only have a few wrinkles. It may be contraindicated. Elderly individuals or individuals receiving treatments may thus hesitate to take advantage of said approach. It is expensive. It is reputed to have a risk of causing undesirable aesthetic effects such as, in particular, the lack of a natural appearance, or even skin sagging or local paralysis effects.

According to a second approach, a surgical procedure is performed, which comprises detaching the skin, pulling on the muscle tissue, cutting a part of the muscle tissue, then resuturing the muscles in the new position and, finally, replacing the skin. This approach has the same drawbacks as the previous one. In addition, and unlike the previous approach, it can only be reversed with great difficulty, or not at all. Thus, the approach can prove to be very problematic if the result obtained is different from the result expected. For all these reasons, it can only be used for highly motivated individuals.

Solutions for camouflaging wrinkles by means of an overall treatment of the entire surface to be treated are known. They comprise covering the skin with a more or less thick overall layer. The term “overall” is intended to mean that the material covers the skin, without being limited to only the hollow formed by the wrinkle.

Since the skin is amply covered, the wrinkles are partly filled in and disappear. This technique, which is sometimes used in the movies, cannot be suitable for a normal makeup process. The result is uncomfortable, especially in the event of perspiration, and the thick layer often does not hold over time. In addition, the aesthetic appearance of the skin is lost. Finally, this technique poses the problem of demarcations. This is because, unless the entire visible skin is covered, the outside observer quickly notices that certain parts of the skin do not have the same appearance as the rest. It is therefore necessary, if an aesthetic result is desired, to treat the thick layer and also the non-covered skin in order to limit the demarcation effects.

There therefore remains a need for new methods for masking marked reliefs, in particular “marked” wrinkles and “medium” wrinkles, and completely or partly solving the problems discussed with reference to the known methods.

An object of the invention is in particular to propose a method which makes it possible to treat all kinds of wrinkles, ranging from slight wrinkles to marked wrinkles.

Another object of the invention is to provide a method which offers a result that is quite long-lasting and has better aesthetics than those obtained with the known methods.

An object of the invention is also to offer a method which has no contraindication, which is not invasive, which is painless, which does not make it necessary to take any risks and which is inexpensive to implement.

These objects, along with others, are achieved by means of a method for smoothing out the skin by filling in recessed portions of skin, said method comprising filling the recessed portions by means of a filling-in product which has, when it is applied in said recessed portions, an elastic modulus of greater than 20 000 Pascals at 25° C. for a stress frequency of 1 Hertz, a flow point of greater than 500 Pascals and, for applied stresses of less than the yield stress, a viscosity of greater than 100 000 Pa.s at a shear rate of 10⁻³s⁻¹, the product being such that, once applied in said recessed portions of the skin, its mechanical properties change subsequent to at least one reaction other than an evaporation reaction.

Advantageously, this is a reaction other than a laser polymerization reaction.

Thus, the method according to the invention is based on filling in the hollowed areas by means of a filling material having particular rheological properties. The term “filling in” is intended to mean, not simply depositing a layer which moulds the profile of the wrinkle and which fills only the bottom thereof, but, on the contrary, filling at least a large part of, preferably to the brim, the recessed area to be masked, in particular the wrinkles, the pores or the folds.

Preferably, the depositing of filling-in composition is limited so as not to spread beyond the recessed area.

However, it is sometimes difficult not to go beyond the hollow to be filled in. In this case, the procedure will be such that the deposit outside the wrinkles is very very thin: typically less than 25% of the thickness of the material in the wrinkle. Also in this case, the procedure will be such that the surface covered outside the wrinkle is limited. The procedure will, for example, be such that the filling-in material does not extend beyond one centimetre over the side of the wrinkles, and even more preferably less than 5 mm over the side of the wrinkles.

The fact of going beyond the wrinkle may not constitute a major problem if the filling-in composition does not have cohesive mechanical properties. Thus, if the material which lies beyond the wrinkle flows or is liquid or is very soft or if it is destructured (in the form of powder or small pieces), it will not exhibit a maintaining force acting on the material included in the wrinkle.

It can also be envisaged to cut the film beyond the wrinkle, for example, by using a tool which cuts the material on the edge of the wrinkles.

According to an essential characteristic of the invention, the mechanical properties of the filling-in material, in particular its rigidity and/or its elasticity, change after having been deposited in the wrinkle.

In the case where not all the product is located in the wrinkle, the change in the material which is not in the wrinkle may be identical to or different from that of the material which is in the wrinkle.

By way of example, the material outside the wrinkle may not change.

According to another example, the material in the wrinkle may change so as to become more elastomeric, whereas the material outside the wrinkle may change so as to become more crumbly or brittle.

According to yet another example, the material in the wrinkle may change so as to become more insoluble (in addition to the variations in its mechanical properties), whereas the material outside the wrinkle may remain soluble.

The change in the mechanical properties of the filling-in product (just like the other optional changes) may be natural or triggered.

The implementation of the method according to the invention results in a good hold over time, a skin which appears to be natural, does not have a covered appearance, has no feeling of discomfort, and does not have a fixed appearance.

The method according to the invention can be used in particular on marked wrinkles and medium wrinkles, and also on the other reliefs of the skin, such as stretch marks, scars, folds and dilated pores.

In addition to the masking of wrinkles, the implementation of the method according to the invention makes it possible to support the skin and reduce the forces and movements which are often concentrated on the wrinkles, this being the case, as mentioned above, without the skin being fixed as a whole. This support will enable there to be less stress placed on the skin and may therefore limit cracking of the skin.

In addition, the method according to the invention may have an action on the wrinkles which form and which, although barely visible, could develop over time. In this respect, the invention makes it possible to have a preventive action.

Finally, makeup removal is facilitated.

### Rheology of the Filling-In Product When Applied to the Portions to be Treated:

The filling-in product used in the method according to the present invention is typically in the form of a paste.

The term “paste” is here intended to mean a product or a composition for which the viscosity can be measured, as opposed to the solid structure of a wand or stick, the viscosity of which cannot be measured.

The products or compositions used for implementing the method according to the invention have characteristic rheological parameters comprising the flow point, the elastic modulus, the viscous modulus and the viscosity.

The flow point or destructuring yield stress τ0 of the filling-in product is defined as being the pressure required to cause a macroscopic flow of the product; it can be determined by performing a stress sweep, for example using a Haake CS150 controlled stress rheometer at a temperature of 25° C.

Preferably, the filling-in product used for implementing the method according to the invention has a flow point of greater than 500 Pascal.

Moreover, the elastic modulus of the filling-in product can be measured for a stress frequency of 1 Hertz, in the “linear viscoelasticity” zone defined by the fact that the stress applied during the measurement is lower than the destructuring yield stress of the composition.

Advantageously, the filling-in product has an elastic modulus of greater than 20 000 Pascal and a viscous modulus of greater than 5000 Pascal.

The viscosity of the filling-in product used for implementing the method according to the invention is preferably greater than 20 000 Pa.s for a shear rate equal to 10⁻³s⁻¹, and preferably greater than 100 000 Pa.s, and in particular less than 20 000 000 Pa.s. The viscosity is measured at 25° C. with a Haake Rheostress RS 150 instrument in the cone-plate configuration under an applied stress.

With regard to their viscosity, the compositions according to the invention exhibit advantageous deformability, elasticity and manageability, making it possible to carry out modelling of the skin, in particular of the facial skin, which is therefore effective for reducing the cutaneous microrelief thereof.

### Change in the Mechanical Properties of the Filling-In Material:

The filling-in material used in the method according to the invention is chosen such that its mechanical properties change. The material may become more rigid or, conversely, become more elastic than at the time when it is applied in the recessed portion to be treated.

The change in the mechanical properties of the filling-in product is such that at least one parameter chosen from the elastic modulus, the flow point, and the viscosity for applied stresses of less than the yield stress, change (preferably increase) by a factor at least equal to 5, and preferably at least equal to 8.

The change in the mechanical properties of the filling-in material can also be monitored by means of measuring the extension before break and the work supplied in order to produce a break by extension. This approach is particularly suitable for the most pasty materials. When the material undergoes a slow mechanical tensile force (1 mm/s, for example), the extension that it can be subjected to before break and also the force necessary to produce this extension are measured.

The change in the filling-in material is preferably such that the extension before break and also the work supplied to produce a break at extension are modified, preferably increased, by a factor at least equal to 2.

For the purposes of the present invention, the targeted change is the result of a reaction other than a reaction of evaporation of a possible volatile phase. The filling-in material can, however, in addition to the change targeted by the invention, undergo a change owing to the simple evaporation of the volatile phase.

Advantageously, it is a reaction other than a laser polymerization reaction.

By way of examples, this change may originate from a (free radical, anionic, etc.) polymerization reaction.

Alternatively, it may be crosslinking by reaction between the functions of two components or crosslinking so as to link, by means of a third component, two functions of two components (polycycloaddition, crosslinking by dimerization).

It may also involve other chemical reactions such as condensations (sol-gels), oxidations (thiols, for example), etc.

The reactions targeted by the invention preferably lead to the creation of covalent bonds or to a rearrangement of covalent bonds.

The change in the mechanical properties may be in the sense of an increase in the rigidity and in the elasticity of the material. Such an increase enables an optimum hold of the filling-in material.

The change in the mechanical properties of the filling-in product may occur naturally or be triggered in response to a stimulus, in particular an energy input.

### 1.—Chemical Crosslinking:

The term “chemical crosslinking” is intended to mean the fact that a compound can, either alone, or by reaction with a second compound, or by the action of radiation or of an energy input, create covalent chemical bonds between the molecules. The result is increased cohesion of the material comprising this compound.

The compound may be a simple molecule or may already be the result of the combination of several molecules, for example oligomers or polymers. The compound may bear one or more reactive functions.

Molecules which, after crosslinking, give a solid and/or deformable but elastomeric material are preferred.

The chemical functions may react with another function of the same nature or react with another chemical function.

1.1.—Reaction with Another Function of the Same Nature:

The functions are, for example, ethylenic functions, in particular acrylate, acrylic, methacrylate, methacrylic or styrene functions.

These molecules generally require an external form of activation in order to react, for example light, heat, the application of a catalyst, or a combination with photoinitiators and, optionally, photosensitizers intended to broaden the range of action of the photoinitiators. Photopolymerizable and/or photocross-linkable compositions are described, for example, in patents CA 1 306 954 and U.S. Pat. No. 5 456 905.

The polymeric compounds bearing ethylenic reactive functions described in patent EP 1 247 515 can be used.

The ethylenic functions can be activated by means of a withdrawing group so as to accelerate the reactions and make the provision of an external activation needless. This is typically the case of the ethyl cyanoacrylate monomer, for which the sole presence of a catalyst such as water allows the reaction.

The ethylenic functions can be activated moderately, for example by means of a withdrawing group. The advantage is that the reaction requires an external activation, which is advantageous for controlling the initiation and the yield of the reaction, but does not require a photoinitiator. For example, this involves cyanoacrylate monomers, and in particular cyanoacrylate monomers in which the group borne by the ester function contains at least 2, if possible 4, carbon-based chains.

Molecules requiring an external activation such as light, but not requiring a photoinitiator, are appreciated. Thus, molecules that are especially preferred are molecules capable of reaction by photodimerization, such as those described in patent EP 1 572 139, in particular those bearing functions such as:

1) stilbazoliums

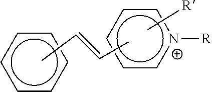

where
R represents a hydrogen atom, or an alkyl or hydroxyalkyl group, and
R′ represents a hydrogen atom or an alkyl group.

The stilbazolium radical is preferably copolymerized with a macromer of poly(vinyl) acetate type (PVA-SBQ). The degree of polymerization of the PVA is between 100 and 5000 and the degree of substitution, as %, of SBQ is between 0.1 and 15.

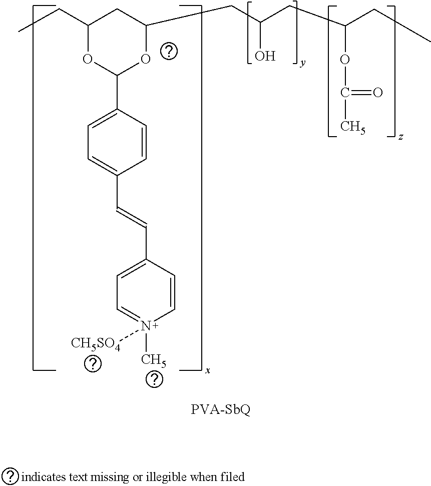

It is possible to add, to these copolymers, particles of PVA or PEVA (PolyEthylene Vinyl Acetate) and/or plasticizers (glycerol, sorbitol, PPG, PBG, DiPG, etc.) in order to modify the mechanical properties thereof.

These materials are particularly appreciated since they do not require a photoinitiator and react at a low UV (UV visible light) dose;

2) styrylazoliums:

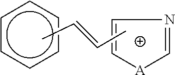

where

A denotes a sulphur atom, an oxygen atom, or an NR′ or C(R′)2 group, R and R′ being as defined above;

3) chalcone;

4) (thio)cinnamate and (thio)cinnamamide;

5) maleimide;

6) (thio)coumarin;

7) thymine;

8) uracil;

9) butadiene;

10) anthracene;

11) pyridone;

12) pyrrolizinone;

13) acridizinium salts;

14) furanone;

15) phenylbenzoxazole;

16) styrylpyrazine.

The reactions which are carried out with another function of the same nature are not limited to reactions involving ethylenic functions.

Compounds that can react by condensation are also appreciated, such as:
siloxane groups, and in particular dialkoxysilane or dihydroxysilane functions, or trialkoxysilane or trihydroxysilane functions. Use may be made of molecules bearing alkyltrialkoxysilane or dialkyltrialkoxysilane functions, and in particular alkylalkoxysilane functions where the alkyl group bears a water-soluble function such as an amine, for example a molecule such as aminotriethoxysilane or aminotriethoxysilane or molecules bearing such functions. In addition to the small siloxane-based molecules (monomers or oligomers), use may be made of compounds which have a higher mass, in particular those described in patent FR 2 910 315;
titanium-based sol-gels.

Derivatives of cinnamic acid which crosslink, under UV excitation, according to a polycycloaddition reaction, are also appreciated. By way of example, mention will be made of polydimethylsiloxane cinnamate, polyvinyl cinnamate copolymers of which the degree of substitution is between 1% and 50%.

With these molecules, the initiation and the yield of the reaction can be controlled.

Compounds that can react by oxidation are also appreciated, such as aromatic compounds bearing at least two hydroxyl functions, or one hydroxyl function and one amine function, or one hydroxyl function, for example catechol or dihydroxyindole. The oxidizing agent may be oxygen from the air or another oxidizing agent, such as aqueous hydrogen peroxide, for example.

1.2. -Reaction with Another Function of Different Nature:

The molecules which react in this case have two types of functions, which are complementary. They may be systems where molecules bearing FA functions and molecules bearing FB functions, which can react with the FA functions, are brought into contact.

They may also be molecules bearing, on the same structure, one or more FA functions and one or more FB functions.

The FA function may be chosen, for example, from:
epoxide,
aziridine,
vinyl and activated vinyl, in particular acrylonitrile, and acrylic and methacrylic esters,
crotonic acid and esters, cinnamic acid and esters, styrene and derivatives, butadiene,
vinyl ethers, vinyl ketone, maleic esters, vinyl sulphones, maleimides,
acid chloride, anhydride and carboxylic acid esters,
aldehydes,
acetals, hemiacetals,
aminals, hemiaminals,
ketones, alpha-hydroxyketones, alpha-haloketones,
lactones, thiolactones,
isocyanate,
thiocyanate,
imines,
imides, in particular succinimide, glutimide,
N-hydroxysuccinimide esters,
imidates,
thiosulphate,
oxazine and oxazoline,
oxazinium and oxazolinium,
C₁ to C₃₀ alkyl halides or C₆ to C₃₀ aryl or aralkyl halides, of formula RX, with X=I, Br or Cl,
halides of unsaturated carbon-based rings or heterocycles, in particular chlorotriazines,
chloropyrimidine, chloroquinoxaline, chlorobenzotriazole,
sulphonyl halide: RSO₂Cl or RSO₂F, R being a C₁ to C₃₀ alkyl.

By way of illustration, mention may be made of the molecules bearing functions of the FA group:

methyl vinyl ether/maleic anhydride copolymer, in particular sold, for example, by the company ISP under the name Gantrez,
polyglycidyl methacrylate, in particular sold by Polysciences,
glycidyl polydimethylsiloxane, in particular sold by the company Shinetsu (reference X-2Z-173 FX or DX),
epoxy polyamidoamine, for example sold by the company Hercules under the name Delsette 101, or Kymene 450 from Hercules,
epoxydextran,
polysaccharide polyaldehydes obtained by oxidation of polysaccharides with NaIO₄ (Bioconjugate Techniques; G T Hermanson, Academic Press, 1996).

The FB function can be chosen from XHn functions where X=O, N, S or COO and n=1 or 2, in particular alcohols, amine, thiol and carboxylic acid.

By way of example, mention may be made, as molecules bearing functions of FB type, of:
PAMAM dendrimer, in particular sold by the company Dendritech, DSM, Sigma-Aldrich (Starburst, PAMAM Dendrimer, G(2, O) from the company Dendritech,
dendrimers comprising hydroxyl functions, in particular sold by the company Perstorp, DSM (example: HBP TNP core 2 Generation Perstorp),
PEI (polyethyleneimine), in particular sold by BASF under the name Lupasol,
PEI-Thiol,
polylysine, in particular sold by the company Chisso,
HP cellulose, such as Klucel EF from the company Aqualon,
aminodextran, for example sold by the company Carbomer,
aminocellulose, for example those described in WO 01/25283 from the company BASF,
PVA (polyvinyl acetal), for example Airvol 540 from the company Airproducts Chemical,
amino PVA, for example sold by the company Carbomer,
chitosan.

Also included in this second case are the molecules that can react by means of a hydrosilylation reaction:

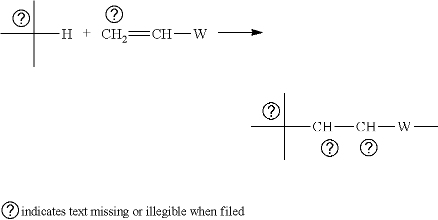

(W represents a carbon-based or silicone chain, for example).

The specifications regarding the two ingredients, the commercially available molecules, the catalyst conditions and the conditions of use are described, for example, in patent application FR 2 910 315.

In one particular case, a molecule already present on the skin, or excreted by the skin, is used as catalysing agent or reactant; typically, water, which can aid the reaction of cyanoacrylates, for example, or certain reactions involving siloxanes.

In another particular case, a molecule present in the ambient air is used as catalysing agent or reactant; typically, oxygen involved in the reaction for crosslinking certain oils, such as drying oils, and in particular drying plant oils such as linseed oil, china wood oil (or tung oil), oiticica oil, vernonia oil, poppy seed oil, pomegranate oil, calendula oil, or alkyd resins. The reactions can be accelerated by the use of catalysts, such as cobalt salts, manganese salts, calcium salts, zirconium salts, zinc salts, strontium salts, lead salts, lithium salts, iron salts, cerium salts, barium salts or tin salts, in the form, for example, of octoate, of linoleate or of octanoate.

In another particular case, molecules which, by rearrangement, will bond to one another, are used. Thus, use may be made of molecules bearing an internal disulphide. New covalent bonds can be created between the molecules by opening of the internal disulphide and reaction of these disulphides.

Catalysts can be used in order to accelerate the reactions; for example, salts of metals such as manganese, copper, iron or platinum, titanates, or enzymes such as oxidases or laccases.

In the case of chemical functions that react with another function of the same or different nature, several methods of application are possible.

For example, all the ingredients that react are integrated into the filling-in composition, or all the ingredients are integrated into the filling-in composition with the exception of one or more compounds, for example either one of the compounds, or a catalyst.

### 2.—Other Possible Reactions:

The change in the properties of the filling-in product can come from a physicochemical phenomenon such as:
coagulation, coacervations or other physicochemical phenomena such as denaturations, complexations or physical crosslinking.

Alternatively, this change can come from a return to equilibrium of the material.

For example, the physical crosslinking can be carried out by means of ingredients capable of creating solid physical bonds between the molecules and conferring water resistance on the final material. These noncovalent bonds are of the ionic or hydrogen type.

By way of example, mention may be made of mixtures with a salt of divalent or polyvalent type, for example of calcium, zinc, strontium or aluminium.

For example a compound A such as an alginate derivative and a compound B such as calcium salt can be mixed. The alginate derivative is, for example, contained in the filling-in composition. An aqueous solution of calcium chloride is applied, in a second step, in the form of a spray for example, in order to bring about the crosslinking.

Mention may also be made of molecules capable of creating strong hydrogen bonds, for instance polysiloxane/polyurea block copolymers, and in particular those of formulae:

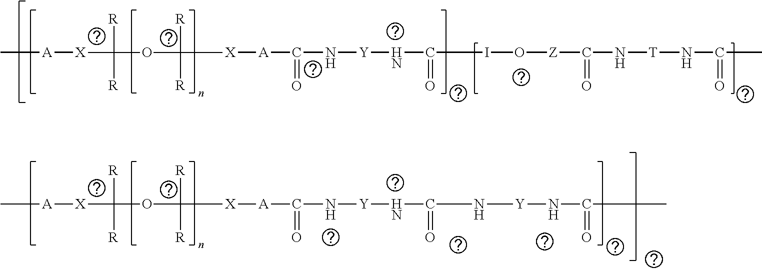

where:

R represents a monovalent hydrocarbon-based radical containing from 1 to 20 carbon atoms, which may be substituted with one or more fluorine or chlorine atoms,
X represents an alkylene radical having 1 to 20 carbon atoms, in which non-neighbouring methylene units may be replaced with —O— radicals,
A represents an oxygen atom or an amino-NR′-radical,
Z represents an oxygen atom or an amino-NR′-radical,
R′ represents hydrogen or an alkyl radical having 1 to 10 carbon atoms,
Y represents a bivalent hydrocarbon-based radical, where appropriate substituted with fluorine or chlorine, having 1 to 20 carbon atoms,
D represents an alkylene radical, where appropriate substituted with fluorine, chlorine, a C₁-C₆ alkyl or a C₁-C₆ alkyl ester, having 1 to 700 carbon atoms, in which non-neighbouring methylene units may be replaced with —O—, —COO—, —OCO— or —OCOO— radicals,
n is a number ranging from 1 to 4000,
a is a number of at least 1,
b is a number ranging from 0 to 40,
c is a number ranging from 0 to 30, and
d is a number greater than 0.

Specifications regarding the functions, the commercially available molecules and the working conditions are given in patent EP 759 812.

The change in the mechanical properties of the filling-in material can occur naturally, i.e. it takes place without any other method once the product has been applied in the wrinkle.

According to a first possibility, the material changes through the action of an element of the environment:
heat, light, etc. (ambient light, ambient humidity, etc.).
in one particular case of the invention, the transformation comes from a natural phenomenon such as perspiration, sebum exudation, etc.

By way of example of this pathway, an aqueous dispersion of partially saponified polyvinyl acetate bearing stilbazolium groups, adsorbed onto polyvinyl acetate particles, is used. This dispersion comprises a solids content of 40%.

Once the material has been applied in the wrinkle, and the edges levelled, it is possible to leave the material to change naturally or to treat it for 1 second with UV A radiation at a rate of 500 mW/cm².

According to a second possibility, the filling-in material changes because a change was initiated during the preparation of the product or during the application and said change continues after the product has been placed in the wrinkle:
a mixing of at least two products at the time of use or on skin,
a transformation or reaction started by agitation or shearing of the product (at the time of use or on the skin).

By way of example, the filling-in material results from preparation by extemporaneous mixing of two compositions, one containing a compound A, the other containing a compound B, at least one of compounds A and B being a silicone compound, and said compounds A and B being capable of reacting together by means of a hydrosilylation, condensation or crosslinking reaction in the presence of a peroxide, when they are brought into contact with one another.

Such technology is described in greater detail in documents WO 01/96450 and GB2407496.

The composition obtained is applied by levelling in the imperfections of the microrelief.

a) Composition Containing Compound A:

| | |
|---|---|
| MICROWAX HW | 0.77 |
| Isononyl isononanoate | 0.46 |
| Suzuki composites | 3.57 |
| Dimethicone | 1.99 |
| Tospearl 145 | 2.87 |
| KSG 6 | 1.65 |
| DOW CORNING 9506 POWDER | 7.18 |
| PSPA (DP100) | 0.13 |
| DOW CORNING 7-FC4210 CURING AGENT | 40 |
| Timiron silk red micro pearlescent agents | 3.57 |
| Nylon fibres | 9.85 |
| Parleam | 5.07 |
| POLYETHYLENE AC 540 | 0.38 |
| Expancel | 0.28 |
| ETHYLHEXYLGLYCERIN | 0.066 |
| CAPRYLYL GLYCOL | 0.066 |
| Microporous silica SB700 | 3.2 |
| Talc | 18.898 |

b) Composition Containing Compound B:

| | |
|---|---|
| MICROWAX HW | 1.86 |
| Isononyl isononanoate | 1.12 |
| Suzuki composites | 3.05 |
| Dimethicone | 2.98 |
| Tospearl 145 | 2.45 |
| KSG 6 | 2.48 |
| DOW CORNING 9506 POWDER | 6.14 |
| PSPA (DP100) | 0.32 |
| DOW CORNING 7-FC4210 ELASTOMER | 40 |
| film-forming base | |
| Timiron silk red micro pearlescent agents | 3.05 |
| Nylon fibres | 8.42 |
| Parleam | 5.99 |
| POLYETHYLENE AC 540 | 0.93 |
| Expancel | 0.24 |
| ETHYLHEXYLGLYCERIN | 0.16 |
| CAPRYLYL GLYCOL | 0.16 |
| Microporous silica SB700 | 2.74 |
| Talc | 17.91 |

These compositions are prepared using a BC21 blender/extruder.

The compositions above are mixed extemporaneously in a 50/50 proportion.

The rheological parameters measured for the composition of the example above, at the time it is applied in the wrinkle, have been reported in Table 1 below:

TABLE 1
Rheological parameters of the composition resulting from the
extemporaneous mixing of compositions a and b of Example 1

| | Extemporaneous | |
|---|---|---|
| | mixture a + b | |
| Elastic modulus G′ at 1 Hertz (Pa) | 630 000 | Pa |
| Viscous modulus G″ at 1 Hertz (Pa) | 115 000 | Pa |
| Flow point τ₀ (Pa) | 1980 | Pa |
| Viscosity (Pa) at 10⁻³ s⁻¹ | 2 × 10⁶ | Pa · s |

This mixture is then applied to the wrinkles and fine lines of the contour of the eyes and the crows feet wrinkles and fine lines of a panel of women with mature skin. A spectacular decrease in the wrinkles and fine lines is then noted in these women.

The hydrosilylation reaction initiated before application of the product in the wrinkles continues after application.

Measurement of the same rheological parameters at t=5 minutes gives the values reported in Table 2 below:

TABLE 2
Rheological parameters of the composition resulting
from the extemporaneous mixing of compositions
a and b of Example 1 at t = 5 minutes

| | Extemporaneous | |
|---|---|---|
| | mixture a + b | |
| Elastic modulus G′ at 1 Hertz (Pa) | >6 000 000 | Pa |
| Viscous modulus G″ at 1 Hertz (Pa) | >1 000 000 | Pa |
| Flow point τ₀ (Pa) | >19 000 | Pa |
| Viscosity (Pa) at 10⁻³ s⁻¹ | >2 × 10⁷ | Pa · s |

These values reflect an increase, by a factor of approximately 10, in each of the rheological parameters of the filling-in product, measured before transition.

According to a third possibility, the material changes since the formula is applied in a form not in equilibrium, which returns to a state of equilibrium once applied:
The compound is fluidified by increasing the temperature. On cooling, the compound resolidifies.
The formula is fluidified by agitation; at rest, it solidifies.

According to a fourth possibility, the material changes since a procedure is followed which makes it possible to initiate the reaction or the transformation once the product has been applied in the wrinkle:
Addition of a third compound which aids the transformation, such as a catalyst, a pH agent, etc.
Addition of a third compound allowing a physicochemical transformation, such as the addition of a solvent or of water, etc., which induces a change in the material.

By way of example, a guar gum powder is used. The powder is introduced into a nonaqueous solvent such as ethanol or acetone.

Once the paste has been applied in the wrinkle, the edges are levelled.

It is allowed to dry, and the wrinkle is moistened with a spray of water containing a phosphate salt.

### Other Ingredients of the Filling-In Product:

a) Active Agents:

The filling-in product according to the invention may contain at least one active agent, in particular intended for treating wrinkles, said active agent being present in the filling-in product prior to application thereof in the recessed portions, or added to the filling-in product after application in the recessed portions of the skin.

The filling-in product used in the method according to the invention may contain one or more cosmetic active agents, and in particular an antiwrinkle active agent.

Examples of antiwrinkle active agents that can be used according to the invention are: retinol and derivatives thereof, such as retinyl palmitate; ascorbic acid and derivatives thereof, such as magnesium ascorbyl phosphate and ascorbyl glucoside; adenosine and derivatives thereof, in particular nonphosphated derivatives thereof; tocopherol and derivatives thereof, such as tocopheryl acetate; nicotinic acid and precursors thereof, such as nicotinamide; ubiquinone; glutathione and precursors thereof, such as L-2-oxothiazolidine-4-carboxylic acid; C-glycoside compounds and derivatives thereof, in particular those described in application WO 02/051828; plant extracts, and in particular extracts of sea fennel and of olive leaf, and also plant proteins and hydrolysates thereof, such as rice or soya protein hydrolysates; algal extracts, in particular of laminaria; bacterial extracts; sapogenins, such as diosgenin and extracts of Dioscorea plants, in particular of wild yam, containing them; α-hydroxy acids; β-hydroxy acids, such as salicylic acid and 5-n-octanoylsalicylic acid; oligopeptides and pseudodipeptides and acylated derivatives thereof, in particular {2-[acetyl(3-trifluoromethylphenyl)amino]-3-methylbutyrylamino}acetic acid and the lipopeptides sold by the company Sederma under the trade names Matrixyl 500 and Matrixyl 3000; lycopene; manganese salts and magnesium salts, in particular gluconates; and mixtures thereof. Mention may also be made of vitamins, for instance vitamins B3 or PP, B5, E and K1.

As active agents most particularly suitable for the invention, mention may in particular be made of retinyl palmitate, tocopherol, tocopheryl acetate, lycopene, and the lipopeptides sold by the company Sederma under the trade names Matrixyl 500 and Matrixyl 3000, and C-glycoside derivatives, and in particular C-β-D-xylopyranoside-2-hydroxypropane.

Preferably, the anti-ageing active agent is chosen from adenosine and derivatives thereof, ascorbic acid and derivatives thereof, and C-glycosides and derivatives thereof such as C-α-D-xylopyranoside-2-hydroxypropane.

The amount of active agents depends, of course, on the nature of the active agent and on the desired effect, but it generally represents from 0.01% to 10% by weight, preferably from 0.1% to 5% by weight, relative to the total weight of the composition.

In addition, the method according to the invention may be used in conjunction with the use of treatments involving the use of waves (visible light, IR, UV, etc.), or of other radiations or energies, such as those derived from electric or magnetic fields, or microwave energies. In these cases, the material can be adapted accordingly. By way of example, it may be an electrical conductor or insulator, or a heat conductor or insulator. It may have physical properties capable of aiding, concentrating or blocking electromagnetic waves.

### Fibres:

The filling-in product of the invention can contain fibres. These fibres, through entanglement, provide the filling-in material with good cohesion.

The term “fibre” should be understood to mean an object of length L and of diameter D such that L is greater than D, and preferably much greater than D, D being the diameter of the circle in which the cross section of the fibre lies. In particular, the L/D ratio (or shape factor) is chosen in the range of from 3.5 to 2500, preferably from 5 to 500, and better still from 5 to 150.

The fibres that can be used in the composition of the invention may be fibres of synthetic or natural, and inorganic or organic origin, and they may be flexible or rigid.

They may be short or long, individual or organized, for example braided.

They may have any shape, and may in particular have a circular or polygonal (square, hexagonal or octagonal) cross section, depending on the specific use envisaged.

The fibres that can be used in the composition according to the invention are preferably chosen from polyamide fibres, cellulose fibres, polyethylene fibres and mixtures thereof. Their length may range from 0.1 to 5 mm, preferably from 0.25 to 1.6 mm, and their average diameter may range from 5 to 50 μm.

The fibres may be those used in the manufacture of textiles, and in particular silk fibre, cotton fibre, wool fibre, flax fibre, cellulose fibre extracted, for example, from wood, from plants or from algae, polyamide fibre (Nylon®, in particular under the names Nylon 6=Polyamide 6; Nylon 6,6 or Nylon 66=Polyamide 6,6; Nylon 12=Polyamide 12), rayon fibre, viscose fibre, acetate fibre, in particular rayon acetate fibre, cellulose acetate fibre or silk acetate fibre, poly(p-phenyleneterephthalamide) fibre, acrylic polymer fibre, in particular polymethyl methacrylate fibre or poly(2-hydroxyethyl methacrylate) fibre, polyolefin fibre, and in particular polyethylene or polypropylene fibre, glass fibre, silica fibre, carbon fibre, in particular in graphite form, polytetrafluoroethylene (such as Teflon®) fibre, insoluble collagen fibre, polyester fibre, polyvinyl chloride fibre or polyvinylidene chloride fibre, polyvinyl alcohol fibre, polyacrylonitrile fibre, chitosan fibre, polyurethane fibre, polyethylene phthalate fibre, and fibres formed from a blend of polymers such as those mentioned above, for instance trilobed polyamide/polyester fibres, and mixtures of these fibres.

The fibres used in surgery may also be used, for instance the resorbable synthetic fibres prepared from glycolic acid and caprolactone (Monocryl from the company Johnson & Johnson); resorbable synthetic fibres of the lactic acid/glycolic acid copolymer type (Vicryl from the company Johnson & Johnson); terephthalic polyester fibres (Ethibond from the company Johnson & Johnson); and stainless steel threads (Acier from the company Johnson & Johnson).

Moreover, the fibres may be treated or untreated at the surface, and coated or uncoated. As coated fibres that can be used in the invention, mention may be made of polyamide fibres coated with copper sulphide to give an antistatic effect (for example, R-STAT fibres from the company Rhodia) or fibres coated with another polymer enabling a particular organization of the fibres (specific surface treatment) or a surface treatment inducing colour/hologram effects (Lurex fibre from the company Sildorex, for example).

The fibres that can be used in the composition according to the invention are preferably chosen from polyamide fibres, cellulose fibres and polyethylene fibres, and mixtures thereof. The length thereof may range from 0.1 to 5 mm, preferably from 0.25 to 1.6 mm, and the average diameter thereof may range from 5 to 50 μm.

Fibres chosen from Nylon 6 (or Polyamide 6), Nylon 6,6 or Nylon 66 (or Polyamide 6,6) and Nylon 12 (or Polyamide 12) fibres, and mixtures thereof, are most particularly suitable.

In particular, use may be made of the polyamide fibres sold by Etablissements P. Bonte under the name Polyamide 0.9 Dtex 0.3 mm (INCI name: Nylon 6,6), having an average diameter of 6 μm, a weight of approximately 0.9 dtex and a length ranging from 0.3 mm to 3 mm, or alternatively the polyamide fibres sold under the name Fiberlon 931-D1-S by the company LCW, having a yarn count of approximately 0.9 dtex and a length of approximately 0.3 mm. Use may also be made of Nylon-66 fibres, having a yarn count of approximately 2 dtex, and a length of approximately 0.3 mm, sold under the name Polyamide brillante trilobée by the company Utexbel (INCI name: Nylon-66).

Use may also be made of cellulose (or rayon) fibres having an average diameter of 50 μm and a length ranging from 0.5 mm to 6 mm, such as those sold under the name Natural rayon flock fibre RC1BE-N003-M04 by the company Claremont Flock. Use may also be made of polyethylene fibres such as those sold under the name Shurt Stuff 13 099 F by the company Mini Fibers.

Advantageously, the filling-in product contains at least 5% by weight of fibres, relative to the total weight of the product.

### Other Fillers:

The other fillers may be of any form, plate-shaped, spherical, hemispherical or oblong, irrespective of the crystallographic form (for example, lamellar, cubic, hexagonal, orthorhombic, etc.).

By way of illustration of these fillers, mention may be made of talc, mica, silica, kaolin, poly-β-alanine powder and polyethylene powder, tetrafluoroethylene polymer (Teflon®) powders, lauroyllysine, starch, boron nitride, hollow polymeric microspheres such as those of polyvinylidene chloride/acrylonitrile, for instance Expancel® (Nobel Industrie), of acrylic acid copolymers, silicone resin microbeads (Tospearls® from Toshiba, for example), elastomeric polyorganosiloxane particles, precipitated calcium carbonate, magnesium carbonate, magnesium hydrogen carbonate, hydroxyapatite, barium sulphate, aluminium oxides, polyurethane powders, composite fillers, hollow silica microspheres, and glass or ceramic microcapsules. Use may also be made of particles, which have the form of portions of hollow spheres, as described in patent applications JP-2003 128 788 and JP-2000 191 789.

### Colorant/Optical Effect Material:

The filling-in product may contain at least one colorant or at least one material capable of modifying an optical property, in particular the gloss, thereof.

a) Interference Particles

The filling-in material used according to the invention may advantageously comprise interference particles, for instance small pearlescent agents or interference pigments.

This is because such interference particles may together give the skin, coated with a film of a composition of the invention, a supplementary effect which lightens the skin, makes it uniform, or even camouflages the skin imperfections. Thus, by virtue of their presence, they advantageously make it possible to reinforce the visual perception of an improved surface appearance of the skin provided by the compositions under consideration according to the invention.

In particular, combined with the fillers, as defined above, the interference particles according to the invention may make it possible to obtain an effect of transparency such that the final effect provided on the skin has virtually no effect, or even no effect at all, on the natural flesh tone of the skin.

For the purpose of the present invention, the expression “interference particle” denotes a particle generally having a multilayer structure such that it allows the creation of a colour effect by interference of light rays, which diffract and scatter differently according to the nature of the layers. Thus, these particles may have colours that vary according to the angle of observation and the incidence of the light. The colour effects obtained are associated with the multilayer structure of these particles and are derived from the physical laws of thin film optics, as, for example, described in Pearl Lustre Pigments—Physical principles, properties, applications R. Maisch, M. Weigand. Verlag Moderne Industrie.

For the purpose of the present invention, the term “a multilayer structure” is intended to denote without distinction a structure formed from a substrate covered with a single layer or a structure formed from a substrate covered with at least two or even several consecutive layers.

The multilayer structure may thus comprise one, or even at least two, layer(s), each layer, optionally independently of the other layer(s), being made of at least one material chosen from the group constituted of the following materials: MgF₂, CeF₃, ZnS, ZnSe, Si, SiO₂, Ge, Te, Fe₂O₃, Pt, Va, Al₂O₃, MgO, Y₂O₃, S₂O₃, SiO, HfO₂, ZrO₂, CeO₂, Nb₂O₅, Ta₂O₅, TiO₂, Ag, Al, Au, Cu, Rb, Ti, Ta, W, Zn, MoS₂, cryolite, alloys, polymers and combinations thereof.

Generally, the multilayer structure is of inorganic nature.

More particularly, the interference particles under consideration according to the invention may be interference pigments, or else pearlescent agents.

The interference particles according to the invention may have a volume-average size of generally less than 40 μm, especially ranging from 0.5 to 40 μm, more particularly less than 30 μm, especially less than 20 μm.

It is understood that the choice of these interference particles is made in such a way as to be, moreover, compatible with the demands in terms of filling required according to the invention. In general, these interference particles are present in an amount sufficient to obtain a homogeneous effect in terms of colouring while at the same time preserving the natural flesh tone of the skin and/or of the lips.

Pearlescent agents are most particularly suitable for the invention.

b) Pearlescent Agents

The term “pearlescent agents” should be understood to mean iridescent particles of any shape, in particular produced by certain molluscs in their shell, or which have been synthesized.

The natural or synthetic pearlescent agents may be monolayer or multilayer, in particular formed from a natural substrate based, inter alia, on mica and which is coated with one or more layers of metal oxide.

Thus, the pearlescent agents may be chosen from white pearlescent agents, such as mica coated with titanium, or with bismuth oxychloride, coloured pearlescent agents, such as titanium mica coated with iron oxides, with ferric blue, with chromium oxide or with an organic pigment of the abovementioned type, and also bismuth oxychloride-based pearlescent agents.

They may advantageously be chosen from mica/tin oxide/titanium oxide pearlescent agents, for instance those sold under the names Timiron Silk Blue®, Timiron Silk Red®, Timiron Silk Green®, Timiron Silk Gold® and Timiron Super Silk® proposed by the company Merck, and mica/iron oxide/titanium oxide pearlescent agents, for instance the Flamenco Satin Blue®, Flamenco Satin Red® and Flamenco Satin Violet® proposed by the company Engelhard and mixtures thereof.

The pearlescent agents may be present in a composition according to the invention in a content ranging from 0.1% to 50%, preferably from 0.1% to 40% by weight, and preferentially from 0.1% to 30% by weight, relative to the total weight of the composition.

More specifically, these pearlescent agents may represent from 0.1% to 15% by weight, more particularly from 0.1% to 7% by weight, and more particularly from 0.1% to 5% by weight, relative to the total weight of the composition.

In addition to the abovementioned compounds, a composition according to the invention may contain other organic materials of pigment or dye type, or else with a specific optical effect.

These supplementary materials may be present in the compositions according to the invention, in a content ranging from 0.1% to 15%, preferably from 0.5% to 12%, and preferentially from 1% to 10% by weight, relative to their total weight.

c) Material with Colour and/or Optical Effect

The term “dyes” should be understood to mean compounds which are generally organic and which are soluble in fatty substances such as oils or in an aqueous-alcoholic phase.

The fat-soluble dyes may be chosen from Sudan Red, DC Red 17, DC Green 6, β-carotene, Sudan Brown, DC Yellow 11, DC Violet 2, DC Orange 5 and quinoline yellow. The water-soluble dyes are, for example, beetroot juice and methylene blue.

The term “pigments” should be understood to mean inorganic or organic, white or coloured particles of any shape which are insoluble in the composition and intended to colour it.

Among the inorganic pigments, mention may be made of optionally surface-treated titanium dioxide, zirconium oxide or cerium oxide, and also zinc oxide, (black, yellow or red) iron oxide or chromium oxide, for instance those sold by the company Sunpuro under the reference PFX 5 Sunpuro Yellow, and Sunpuro Red iron oxide, manganese violet, ultramarine blue, chromium hydrate and ferric blue, metal powders such as aluminium powder and copper powder. The pigments may also be chosen from nanopigments of metal oxides, such as titanium dioxide, zinc oxide, iron oxide, zirconium oxide or cerium oxide, and mixtures thereof. The term “nanopigments” is intended to mean pigments having an average particle size ranging from 1 nm to 500 nm, and preferably ranging from 10 nm to 100 nm.

Among the organic pigments, mention may be made of carbon black, D & C pigments, and lakes, in particular lakes based on cochineal carmine, barium, strontium, calcium and aluminium.

d) Material with Specific Optical Effect:

An optical effect is different from a simple, conventional hue effect, i.e. a unified and stabilized effect of the kind produced by conventional colorants, such as, for example, monochromatic pigments. For the purpose of the invention, the term “stabilized” signifies absence of an effect of variability of colour with the angle of observation or else in response to a temperature change.

For example, the material capable of providing this effect may be chosen from metallic-glint particles, goniochromatic colouring agents, diffracting pigments, thermochromic agents and optical brighteners.

The metallic-glint particles that can be used in the invention are in particular chosen from:
particles of at least one metal and/or of at least one metal derivative,
particles comprising a single-substance or multi-substance, organic or inorganic substrate, at least partially coated with at least one metallic-glint layer comprising at least one metal and/or at least one metal derivative, and
mixtures of said particles.

Among the metals that may be present in said particles, mention may, for example, be made of Ag, Au, Cu, Al, Ni, Sn, Mg, Cr, Mo, Ti, Zr, Pt, Va, Rb, W, Zn, Ge, Te and Se, and mixtures or alloys thereof. Ag, Au, Cu, Al, Zn, Ni, Mo and Cr, and mixtures or alloys thereof (for example bronzes and brasses) are preferred metals.

The term “metal derivatives” denotes compounds derived from metals, in particular oxides, fluorides, chlorides and sulphides.

By way of illustration of these particles, mention may be made of aluminium particles, such as those sold under the names Starbrite 1200 EAC® by the company Siberline and Metalure® by the company Eckart.

Mention may also be made of metal powders of copper or of alloy mixtures, such as the references 2844 sold by the company Radium Bronze, metal pigments, such as aluminium or bronze, for instance those sold under the name Rotosafe 700 from the company Eckart, the silica-coated aluminium particles sold under the name Visionaire Bright Silver from the company Eckart and the metal alloy particles such as silica-coated bronze (copper and zinc alloy) powders sold under the name Visionaire Bright Natural Gold from the company Eckart.

A composite pigment according to the invention may be composed in particular of particles comprising:
an inorganic core,
at least one at least partial coating with at least one organic colorant.

At least one binder may advantageously participate in the attachment of the organic colorant to the inorganic core. This binder may advantageously act without the formation of covalent bonds.

The particles of composite pigment may have varied shapes. These particles may especially be platelet-shaped or globular, in particular spherical, and may be hollow or solid. The term “platelet-shaped” denotes particles for which the ratio of the largest dimension to the thickness is greater than or equal to 5.

A composite pigment according to the invention may, for example, have a specific surface area of between 1 and 1000 m²/g, especially between 10 and 600 m²/g approximately, and in particular between 20 and 400 m²/g approximately. The specific surface area is the value measured by the BET method.

According to one particular embodiment of the invention, the inorganic core is a titanium oxide.

Titanium oxides, in particular TiO₂, iron oxides, in particular Fe₂O₃, cerium oxide, zinc oxide and aluminium oxide, and silicates, in particular aluminosilicates and borosilicates, are most particularly suitable as inorganic core.

The organic colorant may comprise, for example, organic pigments which may be chosen from the compounds below and mixtures thereof:
cochineal carmine,
organic pigments of azo, anthraquinone, indigoid, xanthene, pyrene, quinoline, triphenylmethane or fluorane dyes,
insoluble organic sodium, potassium, calcium, barium, aluminium, zirconium, strontium or titanium salts or lakes of acid dyes such as azo, anthraquinone, indigoid, xanthene, pyrene, quinoline, triphenylmethane or fluorane dyes, it being possible for these dyes to comprise at least one carboxylic or sulphonic acid group.

Among the organic pigments, mention may in particular be made of those known under the following names: D&C Blue No. 4, D&C Brown No. 1, D&C Green No. 5, D&C Green No. 6, D&C Orange No. 4, D&C Orange No. 5, D&C Orange No. 10, D&C Orange No. 11, D&C Red No. 6, D&C Red No. 7, D&C Red No. 17, D&C Red No. 21, D&C Red No. 22, D&C Red No. 27, D&C Red No. 28, D&C Red No. 30, D&C Red No. 31, D&C Red No. 33, D&C Red No. 34, D&C Red No. 36, D&C Violet No. 2, D&C Yellow No. 7, D&C Yellow No. 8, D&C Yellow No. 10, D&C Yellow No. 11, FD&C Blue No. 1, FD&C Green No. 3, FD&C Red No. 40, FD&C Yellow No. 5 and FD&C Yellow No. 6.

According to one particular embodiment, the D&C Red No. 7 organic pigment is used.

According to another embodiment, the D&C Red No. 28 organic pigment is used.

According to another particular embodiment, the FD&C Yellow No. 5 organic pigment is used.

According to one particular embodiment, the organic FD&C Blue No. 1 aluminium lake is used.

According to another embodiment, the organic FD&C Yellow No. 5 aluminium lake is used.

According to one particular embodiment, the organic binder is a polymethylhydrogensiloxane.

By way of illustration of composite pigments of this type, mention may in particular be made of those composed as follows:
titanium dioxide (CI77891), FD&C Blue aluminium lake (CI42090) and polymethylhydrogensiloxane (58.1/40.7/1.2)
titanium dioxide (CI77891), D&C Red No. 7 (CI15850) and polymethylhydrogensiloxane (65.8/32.9/1.3)
titanium dioxide (CI77891), D&C Red No. 28 (CI45410) and polymethylhydrogensiloxane (65.8/32.9/1.3)
titanium dioxide (CI77891), FD&C Yellow 5 aluminium lake (CI191140) and polymethylhydrogensiloxane (65.8/32.9/1.3).

According to one alternative, a composite pigment suitable for the invention may also be composed of an inorganic core, in which is dispersed at least one organic or inorganic colorant, such as the pigments of Suzuki or Ercolano type.

By way of illustration of composite pigments suitable for the invention, mention may also be made of the pigments distributed under the name PC-LS-14 or PC-LS-19 by the company Miyoshikasei, and also the Rosso Er Colano pigments from the company Dolci Colori.

The particles in question may also be particles comprising a glass substrate, such as those sold by the company Nippon Sheet Glass under the name Microglass Metashine.

The goniochromatic colouring agent may be chosen, for example, from multilayer interference structures and liquid-crystal colouring agents.

Examples of symmetrical multilayer interference structures that can be used in compositions prepared in accordance with the invention are, for example, the following structures: Al/SiO₂/Al/SiO₂/Al, pigments having this structure being sold by the company Dupont De Nemours; Cr/MgF₂/Al/MgF₂/Cr, pigments having this structure being sold under the name Chromaflair by the company Flex; MoS₂/SiO₂/Al/SiO₂/MoS₂; Fe₂O₃/SiO₂/Al/SiO₂/Fe₂O₃ and Fe₂O₃/SiO₂/Fe₂O₃/SiO₂/Fe₂O₃, pigments having these structures being sold under the name Sicopearl by the company BASF; MoS₂/SiO₂/mica-oxide/SiO₂/MoS₂; Fe₂O₃/SiO₂/mica-oxide/SiO₂/Fe₂O₃; TiO₂/SiO₂/TiO₂ and TiO₂/Al₂O₃/TiO₂; SnO/TiO₂/SiO₂/TiO₂/SnO; Fe₂O₃/SiO₂/Fe₂O₃; SnO/mica/TiO₂/SiO₂/TiO₂/mica/SnO, pigments having these structures being sold under the name Xirona by the company Merck (Darmstadt). By way of example, these pigments may be the pigments of silica/titanium oxide/tin oxide structure sold under the name Xirona Magic by the company Merck, the pigments of silica/brown iron oxide structure sold under the name Xirona Indian Summer by the company Merck and the pigments of silica/titanium oxide/mica/tin oxide structure sold under the name Xirona Caribbean Blue by the company Merck. Mention may also be made of the Infinite Colors pigments from the company Shiseido. Depending on the thickness and the nature of the various layers, various effects are obtained. Thus, with the Fe₂O₃/SiO₂/Al/SiO₂/Fe₂O₃ structure, the colour changes from green-golden to red-grey for SiO₂ layers of 320 to 350 nm; from red to golden for SiO₂ layers of 380 to 400 nm; from violet to green for SiO₂ layers of 410 to 420 nm; from copper to red for SiO₂ layers of 430 to 440 nm.

By way of example of pigments with a polymeric multilayer structure, mention may be made of those sold by the company 3M under the name Color Glitter.

Examples of liquid-crystal goniochromatic particles that may be used include those sold by the company Chenix, and also those sold under the name Helicone® HC by the company Wacker.

These materials may be present in a content ranging from 20% to 75% by weight, preferably from 20% to 50%, relative to the total weight of the composition.

### Physiologically Acceptable Medium:

The filling-in material used for implementing the method according to the invention comprises a physiologically acceptable medium, i.e. a nontoxic medium which can be applied to human keratin materials and which has a pleasant appearance, odour and feel.

It may contain at least one liquid fatty phase formed from at least one oil.

According to one embodiment variant, the filling-in products according to the invention may be in an anhydrous form.

For the purpose of the invention, the expression “anhydrous composition” denotes a composition which contains less than 2% by weight of water, or even less than 0.5% of water, relative to its total weight, and in particular a composition free of water.

As examples of oils that can be used in the composition according to the invention, mention may be made of:
hydrocarbon-based oils of animal origin, such as perhydrosqualene;
hydrocarbon-based oils of plant origin, such as liquid triglycerides of fatty acids containing from 4 to 10 carbon atoms, for instance heptanoic or octanoic acid triglycerides or else, for example, sunflower oil, maize oil, soybean oil, marrow oil, grapeseed oil, sesame oil, hazelnut oil, apricot oil, macadamia oil, arara oil, castor oil, avocado oil, caprylic/capric acid triglycerides such as those sold by the company Stearineries Dubois or those sold under the names Miglyol 810, 812 and 818 by the company Dynamit Nobel, jojoba oil, Shea butter oil;
synthetic esters and ethers, in particular of fatty acids, for instance oils of formulae R₁COOR₂ and R₁OR₂ in which R₁ represents the residue of a fatty acid containing from 8 to 29 carbon atoms and R₂ represents a branched or unbranched hydrocarbon-based chain containing from 3 to 30 carbon atoms, for instance purcellin oil, isononyl isononanoate, isopropyl myristate, 2-ethylhexyl palmitate, 2-octyldodecyl stearate, 2-octyldodecyl erucate, isostearyl isostearate, hydroxylated esters such as isostearyl lactate, octyl hydroxystearate, octyldodecyl hydroxystearate, diisostearyl malate, triisocetyl citrate, fatty alcohol heptanoates, octanoates and decanoates; polyol esters, such as propylene glycol dioctanoate, neopentyl glycol diheptanoate and diethylene glycol diisononanoate; and pentaerythritol esters, for instance pentaerythrityl tetraisostearate;
linear or branched hydrocarbons of inorganic or synthetic origin, such as volatile or non-volatile liquid paraffins, and derivatives thereof, isohexadecane, isododecane, petroleum jelly, polydecenes, hydrogenated polyisobutene such as Parleam® oil;
natural or synthetic essential oils such as, for example, eucalyptus oil, lavandin oil, lavender oil, vetiver oil, Litsea cubeba oil, lemon oil, sandalwood oil, rosemary oil, camomile oil, savory oil, nutmeg oil, cinnamon oil, hyssop oil, caraway oil, orange oil, geraniol oil, cade oil and bergamot oil;
fatty alcohols containing from 8 to 26 carbon atoms, such as cetyl alcohol, stearyl alcohol and the mixture thereof (cetylstearyl alcohol), octyldodecanol, 2-butyloctanol, 2-hexyldecanol, 2-undecylpentadecanol, oleyl alcohol or linoleyl alcohol;
partially hydrocarbon-based and/or silicone-based fluoro oils such as those described in document JP-A-2-295912;
silicone oils, such as volatile or non-volatile polydimethylsiloxanes (PDMSs) comprising a linear or cyclic silicone chain, which are liquid or pasty at ambient temperature, in particular cyclopolydimethylsiloxanes (cyclomethicones), such as cyclohexasiloxane and cyclopentasiloxane; polydimethylsiloxanes comprising pendent alkyl, alkoxy or phenyl groups or alkyl, alkoxy or phenyl groups at the end of the silicone chain, which groups contain from 2 to 24 carbon atoms; phenylated silicones, such as phenyl trimethicones, phenyl dimethicones, phenyltrimethylsiloxydiphenylsiloxanes, diphenyl dimethicones, diphenylmethyldiphenyltrisiloxanes, (2-phenylethyl)trimethylsiloxysilicates and polymethylphenylsiloxanes;
and mixtures thereof.

The term “hydrocarbon-based oil” in the list of oils mentioned above is intended to mean any oil containing predominantly carbon and hydrogen atoms, and optionally ester, ether, fluoro, carboxylic acid and/or alcohol groups.

The compositions according to the invention may comprise a volatile oil.

For the purpose of the invention, the term “volatile oil” is intended to mean an oil capable of evaporating on contact with keratin materials in less than one hour, at ambient temperature and atmospheric pressure. The volatile oils of the invention are volatile cosmetic oils which are liquid at ambient temperature and have a non-zero vapour pressure, at ambient temperature and atmospheric pressure, ranging in particular from 0.13 Pa to 40 000 Pa (10⁻³ to 300 mmHg), in particular ranging from 1.3 Pa to 13 000 Pa (0.01 to 100 mmHg), and more particularly ranging from 1.3 Pa to 1300 Pa (0.01 to 10 mmHg).

As volatile oils, mention may be made, inter alia, of cyclic or linear silicones containing from 2 to 6 silicon atoms, such as cyclohexasiloxane, dodecamethylpentasiloxane, decamethyltetrasiloxane, butyltrisiloxane and ethyltrisiloxane. Use may also be made of branched hydrocarbons such as, for example, isododecane, and also volatile perfluoroalkanes such as dodecafluoropentane and tetradecafluorohexane, sold under the names PF 5050® and PF 5060® by the company 3M, and perfluoromorpholine derivatives, such as the 4-trifluoromethylperfluoromorpholine sold under the name PF 5052® by the company 3M.

### Application of the Filling-In Material:

The filling-in product used in the method according to the invention can be applied with precision by means of a tool, in particular a spatula or a micro airbrush. Alternatively, the product can be applied with less precision, and can go slightly beyond the edges of the recessed portion to be treated. In this case, the compound which lies on the sides may be levelled, for example with a tool which will serve as a scraper. The product can also be removed by absorption, drawing off, evaporation or rinsing, for example with a special composition.

Preferably, the filling-in product has a dry material content of greater than or equal to 40%, and preferably greater than or equal to 60%.

The term “dry material” is intended to mean the accumulation of the compounds which do not evaporate at ambient temperature.

### Other Variants:

According to one particular embodiment, prior to the introduction of the filling-in product into the recessed portions to be treated, a composition capable of improving the hold of the filling-in product, in particular an adhesive composition, is applied to all or part of the portions to be treated. For example, a reactive or nonreactive adhesive, a PSA adhesive, an adhesion promoter or a compound capable of reacting with the filling-in material is used.

According to yet another embodiment, after the application of the filling-in product in the recessed portions, all or part of the filling-in product is covered by means of a film (either preformed, or formed in situ) and/or of a composition containing at least one UV-screening agent. The hold of the product in the wrinkle is thus improved.

1. A method for smoothing skin, the method comprising filling in a recessed portion of skin by applying a filling-in product to the recessed portion of skin,
wherein:
the filling-in product has the following mechanical properties:
an elastic modulus of greater than 20 000 Pascals at 25° C. for a stress frequency of 1 Hertz;
a flow point of greater than 500 Pascals; and
a viscosity, for applied stresses of less than the yield stress, of greater than 100 000 Pa.s at a shear rate of 10⁻³s⁻¹; and
once applied to the recessed portion of the skin, at least one mechanical property of the filling-in product changes subsequent to a reaction other than an evaporation reaction or a laser polymerization reaction.

2. The method of claim 1, wherein at least one mechanical property selected from the group consisting of the elastic modulus, the flow point, and the viscosity for applied stresses of less than the yield stress, changes by a factor of at least 5.

3. The method of claim 1, wherein the filling-in product has a viscosity of greater than 500 000 Pa.s.

4. The method of claim 1, wherein the rigidity of the filling-in product increases once applied to the recessed portion of the skin.

5. The method of claim 1, wherein the elasticity of the filling in product increases once applied to the recessed portion of the skin.

6. The method of claim 1, wherein the at least one mechanical property of the filling-in product changes in response to a stimulus.

7. The method of claim 1, wherein the filling-in product comprises an active agent intended for treating wrinkles, said active agent being either present in the filling-in product prior to its application to the recessed portion of the skin, or added to the filling-in product after application to the recessed portion of the skin.

8. The method of claim 1, wherein the filling-in product is applied with a tool.

9. The method of claim 1, wherein the filling-in product has a dry material content of greater than or equal to 40%,

10. The method of claim 1, wherein the filling-in product comprises a fibre.

11. The method of claim 10, wherein the filling-in product comprises at least 5% by weight of the fiber, relative to a total weight of the product.

12. The method of claim 10, wherein the fibre is at least one selected from the group consisting of a polyamide fibre, a cellulose fibre, and a polyethylene fibre.

13. The method of claim 1, wherein:
the reaction is a reaction that creates, modifies, or both, at least one covalent bond, in the presence or absence of a catalyst; and
the reaction is at least one selected from the group consisting of polymerization, a crosslinking, a condensation, a coagulation, a denaturation, a complexation, a coacervation, and an oxidation.

14. The method of claim 1, wherein the filling-in product comprises a colorant or a material capable of modifying an optical property thereof.

15. The method of claim 1, wherein, prior to the applying the filling-in product to the recessed portion of skin, a composition capable of improving the hold of the filling-in product, is applied to all or part of the recessed portion of skin to be treated.

16. The method of claim 1, wherein, after the applying the filling-in product in the recessed portion of skin, all or part of the filling-in product is covered with a film, a composition comprising at least one UV-screening agent, or both.

17. The method of claim 1, wherein at least one mechanical property selected from the group consisting of the elastic modulus, the flow point, and the viscosity for applies stresses of less than the yield stress, changes by a factor of at least 8.

18. The method of claim 1, wherein the filling-in product is applied with a spatula or a microairbrush.

19. The method of claim 1, wherein the filling-in product has a dry material content of greater than or equal to 60%.

20. The method of claim 1, wherein, prior to the applying the filling-in product into the recessed portion of skin, an adhesive composition capable of improving the hold of the filling-in product is applied to all or part of the recessed portion of skin to be treated.
